(19) United States
(12) Patent Application Publication
(10) Pub. No.: US 2011/0303224 A1
(43) Pub. Date: Dec. 15, 2011

(54) SKIN ADHERENT MEDICAL DEVICES

(76) Inventors: ALAN D. WIDGEROW, Irvine, CA (US); David Andrew Sharbaugh, Irvine, CA (US); Howard Daniel Palte, Boca Raton, FL (US)

(21) Appl. No.: 13/107,883

(22) Filed: May 14, 2011

Related U.S. Application Data
(60) Provisional application No. 61/354,207, filed on Jun. 12, 2010.

(51) Int. Cl. A61M 16/00

(52) U.S. Cl. 128/207.18

(57) ABSTRACT

A skin adherent device is presented. The skin adherent device includes a first portion configured for attaching to a first portion of an anatomy. A second portion is configured for attaching to a second portion of the anatomy. A third portion is configured for attaching to a third portion of the anatomy. The second portion and the third portion are disposed on opposing ends of the skin adherent device. The first portion, the second portion and the third portion include a tacky gel material.

### CROSS-REFERENCES TO RELATED APPLICATIONS

This application claims priority from U.S. provisional patent application Ser. No. 61/354,207, filed Jun. 12, 2010, incorporated herein by reference.

### BACKGROUND

1. Field

The embodiments relate to a means of application of medical devices to the skin, where such application provides comfort for patient and ease of use for medical professionals.

2. Description of the Related Art

Traditional methods of application of devices to the skin, such as cannulas, make use of adhesive agents that stick to the skin but leave a tacky residue and cause discomfort when removed. The potential for skin sensitivities and the limitation for adjusting position are current distinct disadvantages.

Current traditional nasal cannula designs have been associated with pressure ulcers, noisy air passage(s) and difficulty in application. Intravenous line attachments on the other hand, have used adhesive agents, such as tape, but these attachments are uncomfortable for the patient especially upon removal.

A nasal cannula is generally used wherever small amounts of supplemental oxygen are required, such as in oxygen therapy and indications such as sedative applications. Most cannulae can only provide oxygen at low flow rates-up to 6 litres per minute (L/min)-delivering an oxygen concentration of 28-44%. Rates above 6 L/min can result in discomfort to the patient, drying of the nasal passages and possibly nose bleeds (epistaxis). Current devices, even at rates less than 6 L/min., often are associated with noise disturbance due to turbulent flow of air within different tube diameters. Variable delivery devices fit into two categories, nasal cannula and facemasks. The premise behind nasal cannula is to use the dead space of the nasopharynx as a reservoir for oxygen. When the patient inspires, entrained air mixes with the reservoir air and the inspired gas is enriched. Obviously, the FiO₂ (fraction of inspired oxygen) depends on the magnitude of flow of oxygen, the patient's minute ventilation and peak flow. For most patients, each additional 1 liter per minute of O2 flow with nasal cannula represents an increase in the FiO₂ by 4%. So one liter is 24%, two liters is 28%, and so on. At six liters (44%), it is not possible to raise the FiO₂ further, due to turbulence, in the tubing and in the airway.

The nasal cannula is often used in elderly patients or patients who can benefit from oxygen therapy. Nasal cannulae may also be used by pilots and passengers in small, unpressurized aircrafts that do not exceed certain altitudes. Additionally high flows of an air/oxygen blend can be administered via a nasal cannula to accurately deliver high concentrations of oxygen. Respiratory gas humidification allows the high flows to be delivered comfortably via the cannula. Nasal high flow can be used as an alternative to face mask oxygen and allows the patient to continue to talk, eat and drink while receiving the therapy.

There are problems with current nasal cannulae, for example, if they are not positioned at the nares (nostrils), they are useless. Disorientated patients appear to be remarkably successful at dislodging cannula. This is even the case where the design includes passage around the ears, under the chin and around the scalp as in most currently designed devices. The advantage of nasal cannula is that the patient can comfortably eat and speak easily while receiving oxygen. In many cases it is necessary to administer the gas for lengthy periods and difficulty has been experienced in supporting the catheter tube or tubes in operative position so as to permit the patient to eat, read, converse or perform other functions with a reasonable degree of comfort. The present methods of attachment of the nasal cannulae usually necessitate movement of the head and neck to allow circumferential application of the tubes with fixation at the back of the scalp. Additionally, the tubes are usually routed around the top of the ears and/or below the chin. This leaves the patient vulnerable to consequences of pressure in these areas with subsequent inflammation, pain and ulceration being reported in many cases.

Oxygen and other gases are often administered to patients for varying reasons. Traditionally this apparatus has consisted of a hollow tube connected to a supply (canister or other) on one end extending to nasal prongs or catheters that are inserted into the nasal orifices (nostrils) on the other end to deliver the gas to the patient. The decrease in cross-sectional area as the tubing transitions from the larger tube at the supply end to two smaller tubes near the delivery end causes the velocity of the airflow to increase resulting in increased noise, and in patient discomfort caused when the flow of the gas can be sensed on the nasal mucosa. Patients have objected both to the noise, and to the discomfort.

Traditional nasal cannula are steadied in place using many alternatives, for example, the tubes may be fastened behind the head by means of a sliding plastic fastener; tubes are normally placed over the ears, and they may also be secured to the forehead with tape. The tubing may be looped over the patient's ears and brought together under the chin by a sliding connector that holds the cannula in place. Many problems have been reported with these applications, such as pressure ulcers on the ears and other areas have resulted; irritation may arise from the use of tape; difficulty is often reported in application of the apparatus in unconscious/semi-conscious patients with significant movement of the head and neck needed for application of the device; difficulty is reported with self application in the infirm and elderly patients; and slippage of nasal prongs from the nostrils is also a problem.

### SUMMARY

One embodiment of the invention provides a skin adherent device. The skin adherent device includes a first portion configured for attaching to a first portion of an anatomy. A second portion is configured for attaching to a second portion of the anatomy. A third portion is configured for attaching to a third portion of the anatomy. The second portion and the third portion are disposed on different sides of the skin adherent device. The first portion, the second portion and the third portion include a tacky gel material.

Another embodiment of the invention provides a cannula device. The cannula device comprising a first portion configured for coupling to an upper lip portion of an anatomy. A second portion is configured for coupling to a second portion of the anatomy. A third portion is configured for coupling to a third portion of the anatomy. The second portion and the third portion are disposed on different sides of the cannula device. The first portion, the second portion and the third portion comprise a tacky gel material.

Yet another embodiment of the invention provides an anchoring device configured for coupling other devices to an anatomy. The anchoring device comprises a tacky gel material.

Other aspects and advantages of the present invention will become apparent from the following detailed description, which, when taken in conjunction with the drawings, illustrate by way of example the principles of the invention.

### BRIEF DESCRIPTION OF THE DRAWINGS

The embodiments are illustrated by way of example, and not by way of limitation, in the Figures of the accompanying drawings and in which like reference numerals refer to similar elements and in which:

FIG. 1 illustrates a perspective view of a skin adherent medical device system shown superimposed on a face according to one embodiment of the invention;

FIG. 2 illustrates a front perspective view of a skin adherent medical device according to one embodiment of the invention;

FIG. 3 illustrates a top view of a skin adherent medical device according to one embodiment of the invention;

FIG. 4 illustrates a front view of a skin adherent medical device according to one embodiment of the invention;

FIG. 5 illustrates a rear view of a skin adherent medical device according to one embodiment of the invention;

FIG. 6 illustrates a perspective view of a tubing anchor device coupled with a skin adherent medical device according to one embodiment of the invention;

FIG. 7 illustrates a front view of a tubing anchor device coupled with a skin adherent medical device according to one embodiment of the invention;

FIG. 8 illustrates a side view of a tubing anchor device coupled with a skin adherent medical device according to one embodiment of the invention;

FIG. 9 illustrates a rear view of a tubing anchor device coupled with a skin adherent medical device according to one embodiment of the invention;

FIG. 10 illustrates a front view of a skin adherent medical device coupled to a right sided tube attachment portion according to one embodiment of the invention;

FIG. 11 illustrates a front view of another skin adherent medical device according to one embodiment of the invention;

FIG. 12 illustrates a front view of yet another skin adherent medical device according to one embodiment of the invention;

FIG. 13 illustrates a graph showing oxygen saturation (%) vs. oxygen flow rate according to one embodiment of the invention; and

FIG. 14 illustrates oxygen saturation with increasing oxygen flows for embodiments of the invention.

### DETAILED DESCRIPTION

The following description is made for the purpose of illustrating the general principles of the invention and is not meant to limit the inventive concepts claimed herein. Further, particular features described herein can be used in combination with other described features in each of the various possible combinations and permutations. Unless otherwise specifically defined herein, all terms are to be given their broadest possible interpretation including meanings implied from the specification as well as meanings understood by those skilled in the art and/or as defined in dictionaries, treatises, etc.

The description may disclose several preferred embodiments of medical application devices, systems and kits, as well as operation and/or component parts thereof. While the following description will be described in terms of medical application systems and devices for clarity and to place the invention in context, it should be kept in mind that the teachings herein may have broad application to all types of systems, devices and applications.

Some embodiments of the invention provide a simple, lightweight nasal inhalation apparatus that does not need to be applied around the circumference of the head, nor looped over the ears, which can be simply applied to the nose and cheek areas by way of hypoallergenic skin contact material such as hydrogel, hydrocolloid or silicone that adheres to the skin without adhesive ‘tacky’ effects. This provides an apparatus that is a single interconnected and integrated unit that is convenient, easy to apply and re-apply for patients and medical staff alike.

Other embodiments of the invention provide for application of intra-venous lines to the skin in peripheral locations or in for central line locations. In one example, an embodiment of the invention is used as a catheter-site anchor and dressing. Catheter-site dressing has generated considerable interest in the literature for decades, yielding debates and contradictory findings. Semi-permeable transparent dressings are currently widely used. These are simple to place, allow continuous observation of the skin insertion site and reduce the risk of extrinsic contamination. The known semi-permeable transparent dressings, however, promote moisture and bacterial proliferation under the cover and have been associated with higher catheter-related infection rates when compared with traditional gauze dressings. Therefore, the use of known semi-permeable transparent dressings cannot be recommended in critically ill patients. In contrast to the known semi-permeable transparent dressings, embodiments of the invention provide the benefits of a comfortable dressing that adheres to the skin without tacky adhesiveness, but additionally, the material provides an ideal balance of minimal fluid absorption with a gentle occlusive environment. This results in a dry surround with a closed environment that prevents organisms from entering the area through the dressing, which results in a marked improvement over current techniques and materials. Thus, embodiments of the invention provide a balanced microenvironment—a minimally moist environment together with absorption of any excess moisture aiding in decreasing proliferation of organisms, providing comfort for application and especially for removal, and still maintaining a transparent characteristic to allow observation of a catheter site.

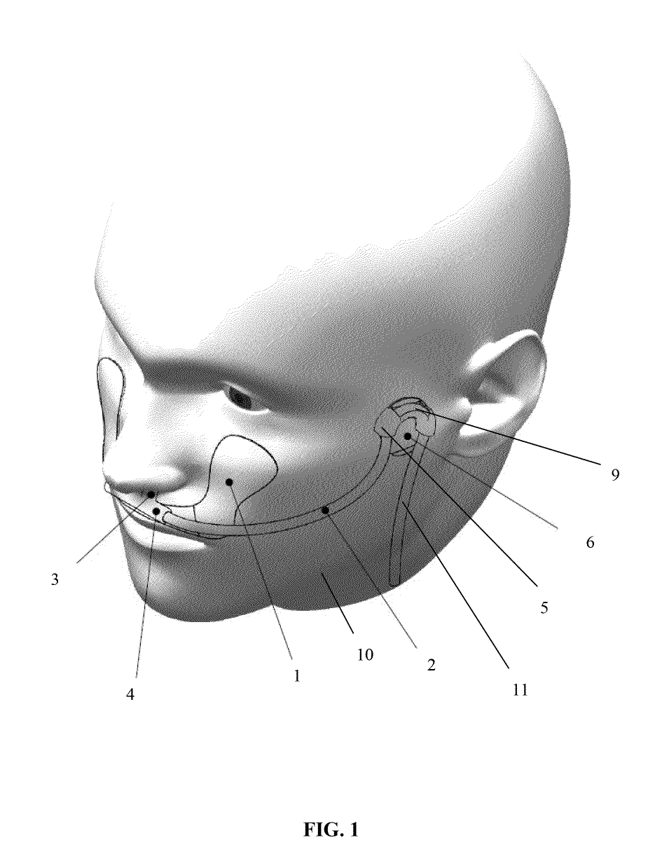

FIG. 1 illustrates a perspective view of a skin adherent medical device system shown superimposed on a face 10 according to one embodiment of the invention. In this embodiment of the invention, the skin adherent medical device system forms a cannula. In one embodiment of the invention, the skin adherent medical device system includes adherent skin patches 1, which attach to the upper lip and paranasal areas. In one example, attached to the anterior aspect of the adherent skin patches 1 is the tubular bridge portion 4 and nasal prongs 3 that deliver gases to the nasal passages. In this example, the gases from the single inlet on the tubular bridge portion 4 are delivered to both nasal prongs 3.

In one example the nasal prongs 3 are curved and directed posteriorly to improve positioning within the nasal passages. In one embodiment of the invention the nasal prongs 3 may also be round or oval in design. In one example, the gas delivery tube 2 is attached to only one side of the tubular bridge portion 4, which is distinguishable from the typical devices that have tubes attached to either side for passing gases to typical straight nasal prongs.

In one example, the tubular bridge portion 4 is adapted for bearing against a person's upper lip and cheeks. In this example, the tubular bridge portion 4 forms the terminating end of a length of tubing 2 through which a gas may be delivered to a patient. The tubular bridge portion 4 has projecting nasal prongs 3 designed in a round or oval, flat comfortable design that accomplish delivery of the gas directly to the patient's nostrils.

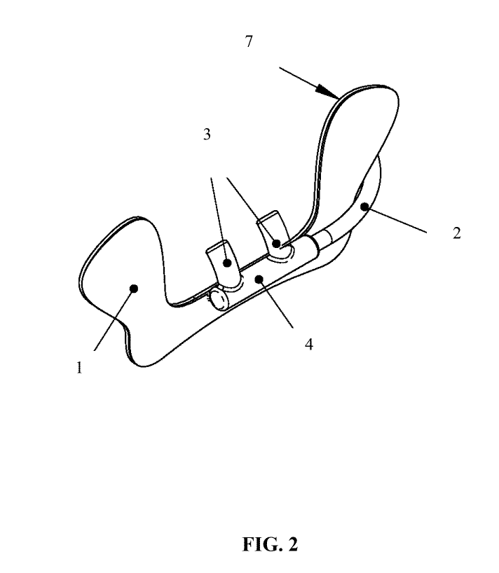

In one embodiment of the invention, the tubular bridge portion 4 may be formed as part of the anterior surface of a paddle portion of the adherent skin patches 1, comprising a flat, flexible surface shaped to fit on the upper lip and around the nose and lie flat against a person's skin. The skin paddle base may comprise material(s), such as hydrogel or equivalent materials, that allow non allergenic hyposensitive application of the device to the lip and paranasal cheek area, which secures the tubular bridge portion 4 with the nasal prongs 3 in the correct position. In one example, the hydrogel material may have an applied layer of adhesive to the inner portion 7 (see FIG. 2) which allows the hydrogel to adhere to the paddle material. In other embodiments, other material may be used separately or in combination, such as hydrocolloid or similar materials.

In one embodiment of the invention, the tubing 2 passes through an anchoring portion comprised of an anchor skin paddle 6 of like material based with the same hydrogel-like material. In one example, the anchor skin paddle 6 is applied to the zygoma (cheek-bone area, left or right) closer to the ear with the overlying tube portion 9 curled in design over a tube support portion 5 such that the tube portion 11 is directed vertically downward toward a gas canister or other gas supply. Since the zygoma moves little, if at all, during speech or chewing (mastication), a stable anchor portion is provided for the tubing portion 2 and tubing portion 11. Additionally, securing the tubing 2 within a few inches of the nose insulates the paddle portion of the adherent skin patches 1 attached near the patient's nose from any force that might be applied to it by movement of the tubing portion 11. In this embodiment of the invention, such an arrangement allows the patient to perform the normal functions (eat, sleep, converse) with prolonged gas administration with little inconvenience and allows simple re-application of the apparatus without pressure being subjected to any anatomic areas. In some embodiments of the invention, application is a simple two-step maneuver, first nasal placement and fixation, and then cheek/temple/zygoma (wherever most comfortable) placement.

In one example, the tubular bridge portion 4 lies along the upper lip just beneath the nose, and the extended nasal prongs 3 are curved backward so as to cause minimum discomfort whilst mounted in position and to ensure the nasal prongs 3 remain in place in the nostrils. In one example, the paddles of the adherent skin patches 1 are secured to a person's skin with material designed specifically for skin contact applications. These materials provide gentle, non-irritating adhesion allowing for easy, painless and non-traumatic removal. In one example, the adherent skin patches 1 may comprise catalyzed silicone gel elastomer or hydrogel material that is reusable and repositionable on a person's skin. The tube portion 2 extends from the nasal prongs 3 in only one direction laterally and not bilaterally as in traditional designs, which lightens the skin adherent medical device system and simplifies attachment of same.

In one embodiment of the invention, the skin adherent medical device system provides for the flow of therapeutic gas from a source of supply (canister, valve, etc.) to a patient. Unlike the embodiments of the invention, typical applications include a single hollow tube connected to the supply that extends toward the patient. In these typical devices, on nearing the patient's head, this larger tube is joined to two smaller tubes which are each passed to the separate sides of the patient's head. These separate tubes are typically passed over the patient's ears so that the device is positioned and retained in place. In the typical application, the two smaller tubes reconnect beneath the patient's nose in a terminating part configured with individual vertical “prongs” or “catheters” to deliver the gas directly into the patient's nasal passages. As gas flows through this traditional device, moving from a single large tube, into two smaller tubes, the cross-sectional area available for gas flow decreases. This decrease accelerates the speed at which the gas flows through the device. Thus, in the typical application, patients can hear the noise caused by the faster flow, and can detect it on their nasal mucosa. Both the noise and the discernable flow are uncomfortable to patients, especially for extended therapies.

In contrast to the typical applications, the embodiments of the invention comprise a single tube that extends from the supply connection to the terminating part (i.e., the nasal prongs 3) coupled to the patient. In one example, the cross-sectional area available for gas flow increases at each transition from supply to patient. Because of the increasing area, the gas flows more slowly as it approaches the patient resulting in lower noise and less stimulation of the nasal mucosa. For some patients, the flow may be silent and undetectable. Table I compares changes to the cross-sectional area of the tubing, from supply to patient, in prior art and embodiments of the invention.

TABLE I
Change in cross-sectional area over delivery length

| | | | Embodiments | | |
|---|---|---|---|---|---|
| | | | of the | | |
| | | Prior Art | invention | | |
| Supply | Inside diameter A | 0.188 | | 0.094 | |
| end | Area of A | 0.028 | 100% | 0.007 | 100% |
| | Inside diameter B | 0.063 | | NA | |
| | Area of B * 2 | 0.006 | 22% | NA | |
| | (two small tubes) | | | | |
| | Area of terminating | 0.012 | 43% | 0.019 | 274% |
| | part | | | | |
| Patient | Area of nasal | 0.016 | 58% | 0.022 | 317% |
| end | prongs | | | | |

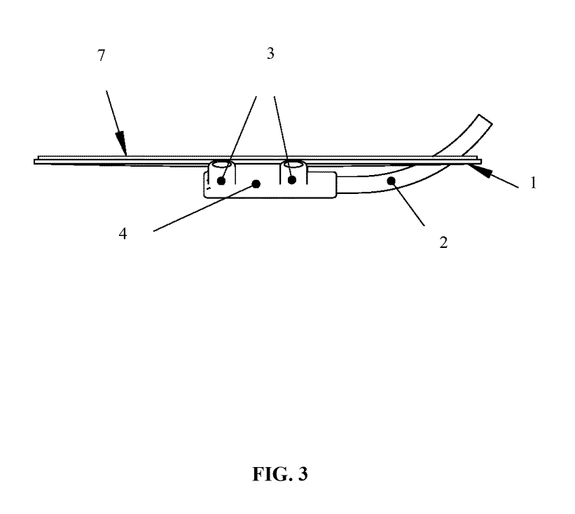

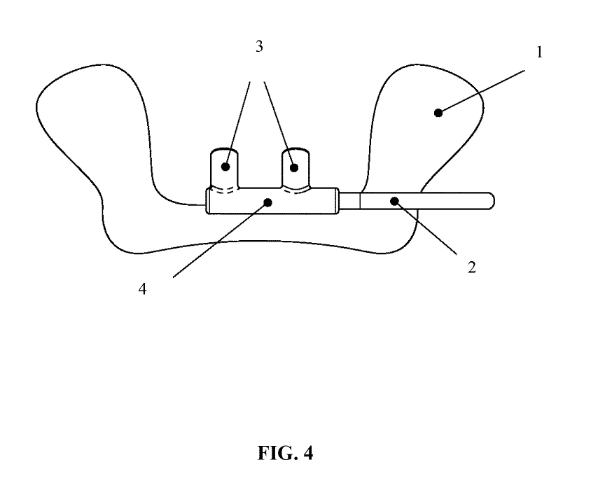

FIG. 2 illustrates an anterior and lateral perspective view of a skin adherent medical device including adherent skin patches 1 comprising adherent non-tacky material such as hydrogel, hydrocolloid, or similar materials. In one example, the tubular bridge portion 4 is configured for extending a tube portion 2 to the left side of a patient's face 10. FIG. 3 illustrates a top view of a skin adherent medical device according to one embodiment of the invention. FIG. 4 illustrates a front view of a skin adherent medical device according to one embodiment of the invention. FIG. 5 illustrates a rear view of a skin adherent medical device according to one embodiment of the invention. In one example, the anterior surface is designed to accommodate the tube portion 2 that extends from the tubular bridge portion 4. The tube portion 2 then traverses a direction upwards and backwards toward a person's ear that is attached with the skin adherent medical device. This anchoring patch fixes the tube portion 2 in a curved position such that the direction of the tube changes from an upward backward direction prior to the coupling to the anchor akin paddle 6 to a vertical downward direction in tube portion 11 (toward the gas canister) after attachment to the tube support portion 5. Therefore, the tubing (i.e., tube portion 2, tube portion 9 and tube portion 11) does not traverse a path over the ears, behind the scalp or under the chin as in traditional prior art designs. In one embodiment of the invention, the tubing, tubular bridge portion 4 and adherent skin patches 1 are integrated in a single system. In another embodiment of the invention, components may be separated and attached to one another in a kit. In other embodiments, different portions (e.g., only patches, specific elements, combinations, etc.) of the components may include hydrogel, hydrocolloid or similar material for adhering to a patient's anatomy. Some embodiments may comprise various combinations of the above-described elements, where some embodiments may comprise less components than others, different combinations, etc.

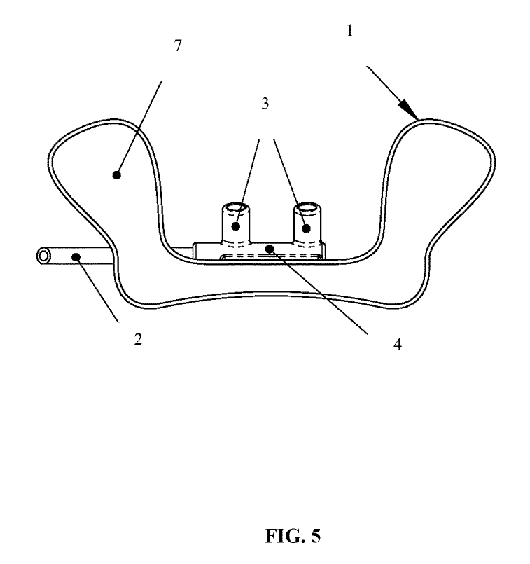

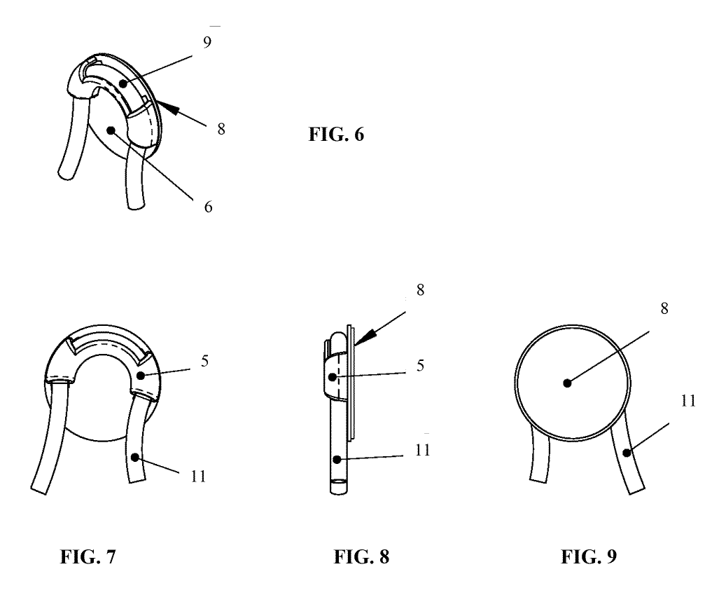

FIG. 6 illustrates a top perspective view of an isolated anchor skin paddle device 6 coupled with a skin adherent medical device 8 according to one embodiment of the invention. FIG. 7 illustrates a front view of the isolated anchor skin paddle device 6 coupled with the skin adherent medical device 8 according to one embodiment of the invention. FIG. 8 illustrates a side view of the isolated anchor skin paddle device 6 coupled with the skin adherent medical device 8 according to one embodiment of the invention. FIG. 9 illustrates a rear view of the isolated anchor skin paddle device 6 coupled with the skin adherent medical device 8 according to one embodiment of the invention. In one example, the anchor skin paddle 6 coupled with the skin adherent medical device 8 is applied to the zygoma (cheek-bone area, left or right) closer to the ear with the overlying tube portion 9 curled in design over a tube support portion 5 such that the tube portion 11 is directed vertically downward toward a gas canister or other gas supply. The inner anchor portion of the skin adherent medical device 8 comprising hydrogel may have an applied layer of adhesive which allows the hydrogel to adhere to the inner portion of the anchor skin paddle device 6.

FIG. 8 illustrates an anterior and lateral perspective view of a skin adherent medical device including adherent skin patches 1 comprising adherent non-tacky material, such as hydrogel or equivalent materials. In one example, the tubular bridge portion 40 is configured for extending a tube portion 2 to the right side of a patient's face 10. In this example, it may be more convenient to have the tubing 2 that extends to the tubing portion 11 traverse the right side of a patient's face 10 due to the layout of a medical facility (e.g., the canister or supply is located to the right of the patient), based on a medical procedure, based on the patient's condition, etc.

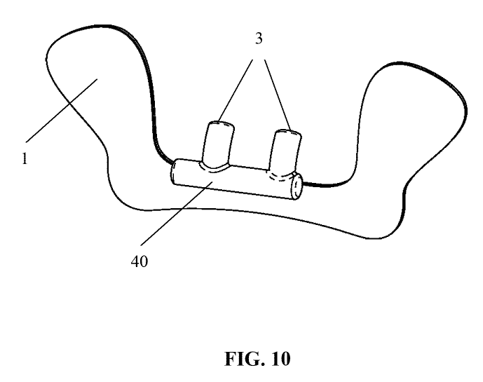

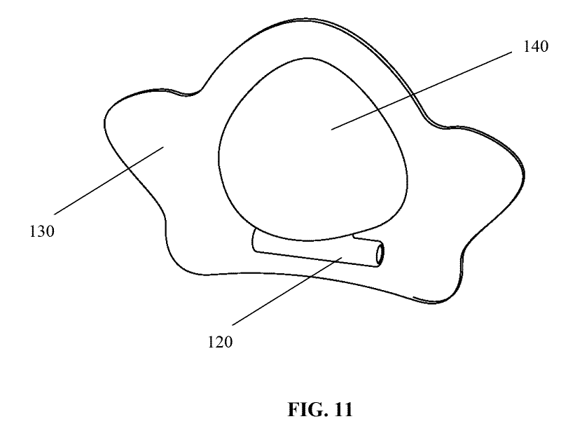

FIG. 11 illustrates a front view of another skin adherent medical device according to one embodiment of the invention. In this embodiment of the invention, the skin adherent medical device includes a nose portion 140 that may be applied around/over a patient's nose for purposes such as dental applications where application to the paranasal cheek area as with the embodiments shown in FIGS. 1-7 and 10 would be prohibitive due to opening and closing of a patient's mouth during procedures/inspection associated with a patient's mouth. The closed nose portion 140 is applicable to closed delivery of gases to a patient's nose, such as nitrous oxide or other gases. Another example of the skin adherent medical device including the nose portion 140 is for continuous positive airway pressure (CPAP) sleeping aids and other closed unit usages.

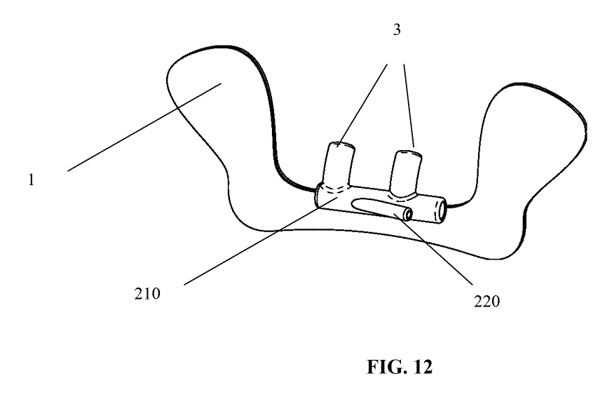

FIG. 12 illustrates a front view of another skin adherent medical device according to one embodiment of the invention. In this embodiment of the invention, the tubular bridge portion 210 includes an additional tube 220. In one example, tube 220 may be used to measure levels such as carbon dioxide via a sensor coupled to tubing attached to tube 220.

### Operation

In some embodiments, the skin adherent medical device, such as a nasal cannula device, is applied by removing a backing from an adhesive base on one side of the upper lip and cheek patch of the adherent skin patches 1. The device is attached, and then the opposite side of the lip and cheek device backing is removed and placed on a patient's skin surface. The nasal prongs 3 are thus placed in position securely adherent to the upper lip and cheek. The second anchor patch backing is then removed. The tubing is adjusted for a comfortable fit and the anchor patch 6 is placed on the skin in the zygoma area. In this example, the entire nasal cannula device is now securely in place. A canister end may then be attached and gas delivery may be initiated.

In one embodiment of the invention, a nasal cannula device comprises a component unit that can be easily applied to the patient without encircling the ears or scalp, which can be anchored to the face without traditional adhesives that cause sensitivities and tackiness, and may be easily re-applied when necessary. In one example, the skin adherent paddles 1 are covered with adherent but not adhesive material such as, but not limited to, hydrogel, silicone, hydrocolloid, or other similar materials. These skin adherent paddles 1 may vary in design but primarily allow fixation to the paranasal and cheek areas with symmetrical or other attachment on either side of the nose and additional paddles where required, typically on the cheek area in front of the ear, on either side of the face. In one example, these skin adherent paddles 1 are joined to the tubing during the manufacturing process such that a single application with simultaneous placement of the skin adherent paddles 1 on the cheek areas and placement of the nasal prongs 3 in the nostril areas allows an easy application of the device.

As an example of the type of material used for the skin adherent paddles 1, a typical specification for the adhesive follows:

| | |
|---|---|
| GEL THICKNESS: | .032″ +/− .004″ |
| LINER 1: | .002″ × 9.75″ BLUE POLYETHYLENE |
| LINER 2: | .005″ × 10″ POLYESTER |
| ELECTRICAL: | IMPEDANCE 200 OHMS MAXIUM AT 10 Hz |

ADHESION: PSTC-5 TEST METHOD: 185 GRAMS/INCH MINIMUM
TO CYTOTOXICITY STUDY USING THE ISO OVERLAY METHOD:
NON-CYTOTOXIC, COMBINED SCORE 0.0
ISO SKIN IRRITATION STUDY ON THE RABBIT:
NON-IRRITATING, COMBINED SCORE 0.0
ISO SENSITIZATION STUDY ON THE GUINEA PIG:
NON-SENSITIZING, COMBINED SCORE 0.0

It should be noted that other embodiments of the invention may include other modifications and changes varied to fit particular operating requirements and environments that will be apparent to those skilled in the art. Therefore, the embodiments of the invention are not considered limited to the examples chosen for purposes of disclosure, and covers all changes and modifications which do not constitute departures from the true spirit and scope of this invention. For example, these variations may include changes in sizes and dimensions for use with children, neonates and infants. The embodiments of the invention may include left and right sided variations. Additional embodiments of the invention may include a ‘closed’ system that completely covers the nose and isolates and seals the nose from the outside air, such as the embodiment of the invention illustrated in FIG. 11. In this example, concentrated inspired volumes of air or gas are allowed to enter the nose in a closed circuit. Therefore, this example may be applicable to dental, CPAP sleeping aids and other closed unit usages.

In other embodiments of the invention, additional applications for the skin adherent medical device material include other shapes, sizes and colors. In one example, a rectangular, flat, transparent patch may be used to anchor intravenous lines (peripheral or central) to the body. In this example, the skin adherent medical device may be applied to the cubital fossa (elbow region), wrist, hand, groin, chest or scalp in adults, children and neonates. In one embodiment of the invention, the skin adherent medical device may be accompanied by a piece of tape that directly places a catheter to a patient's skin where the hydrogel or equivalent material patch may be placed over the catheter with considerable overlap causing the catheter to be securely fixed to the skin. In this example, once the catheter is removed or changed, the adherent patch may be removed painlessly (as opposed to prior art polyurethane film applications). In other examples, such patches may be used on the eyelids, joints, chest and varied anatomic areas for various indications such as post surgical applications, swelling, pain, inflammation (cooled hydrogel patches), anti-allergic, anti-inflammatory, anti-aging and antisepsis (antiseptic, anti-bacterial impregnated hydrogel). Other examples may be used for EKG/ECG (electrocardiogram) electrode applications to a patient's skin or other electrodes or monitoring devices that are typically applied to a patient's skin. It should be noted that unlike traditional devices using adhesives for attachment to a patient's anatomy, the embodiments of the invention may be applied to portions of a patient's anatomy having hair (e.g., facial, arm, chest, etc.) without causing discomfort to the patient upon removing from the portions of the anatomy having hair.

### A Random Volunteer Study Assessing Efficacy of Oxygen Delivery by a New Hydrogel-Adhesive Nasal Oxygen Cannula Medical Device

Supplementary oxygen delivery is required in a host of clinical scenarios. These may be either acute (monitored anesthesia care) or chronic (domiciliary/nocturnal). Blood gas analysis is the gold standard for assessment of oxygenation but is invasive and carries risk. Pulse oximetry affords an inexpensive, non-invasive means of assessing trends in peripheral oxygenation because its paradigm is based on light absorption of two specific light wavelengths that correspond to oxygenated and de-oxygenated hemoglobin (oxygen carrying blood pigment.). Arterial oxygen tensions may be estimated using the Ideal Alveolar Gas Equation. The fraction of inspired oxygen (FiO₂) is an integral and decisive component of this equation. It should be noted that 100% inspired oxygen has an assigned value of 1.0.

### Method

One example of the skin adherent medical device (e.g., a hydrogel-adhesive nasal cannula) was applied to the faces of ten healthy volunteers (5 male & 5 female) in the described manner. The volunteers were placed in a warm environment and lay in the supine position with a pulse oximeter probe attached to the right index finger. No medications were administered throughout the monitoring period. The peripheral oxygen saturation was recorded at 30-second intervals. The total monitoring time was ten minutes. This period was divided into two-minute intervals during which the subjects sequentially breathed in room air, and then supplemental nasal oxygen at flow rates of 2 liters/minute, 3 liters/minute, and 4 liters/minute. In the final two-minute period the subjects breathed room air.

The anticipated FiO₂ for the different intervals are:

| | |
|---|---|
| Room air | 0.21 |
| 21/min | 0.28 |
| 31/min | 0.32 |
| 41/min | 0.36 |

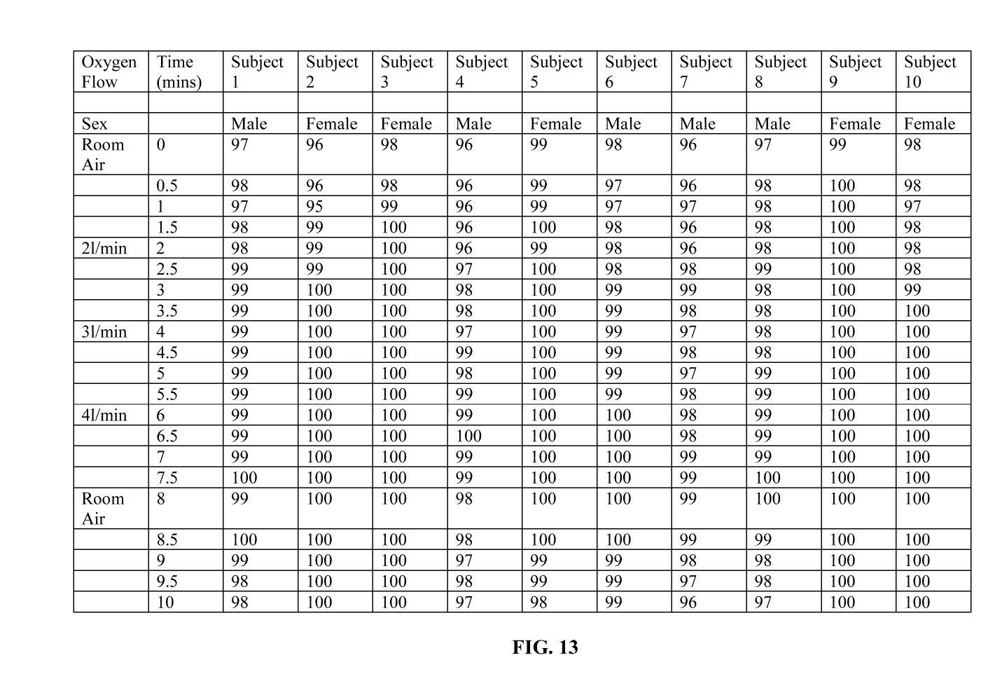

This ratio increases by approximately 0.04 up to a flow rate of about 61/minute, after which physiologic and physical considerations limit the maximum FiO₂ to around 0.5. FIG. 13 provides peripheral % saturation data at varying oxygen flow rates.

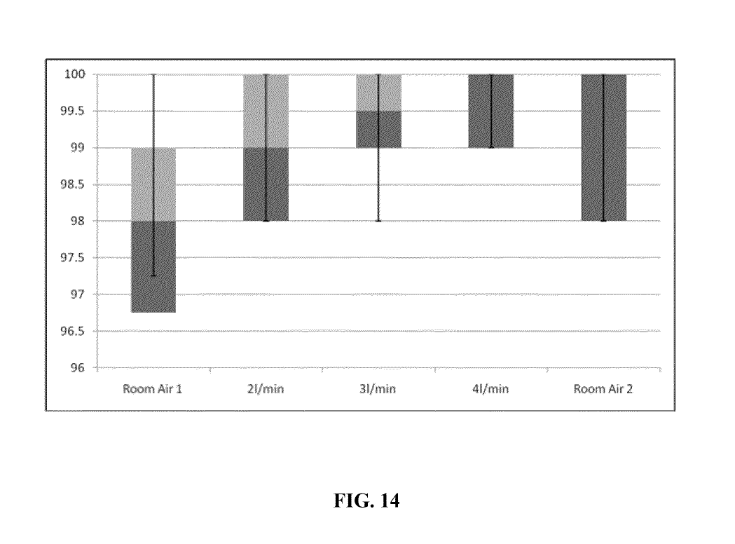

FIG. 14 illustrates an improvement in peripheral oxygen saturation with increasing oxygen flows (or FiO₂) for the embodiments of the invention illustrated in FIGS. 1-5 and 10. The absolute values increase and inter-quartile ranges shrink as oxygen flow rates increase. These indicate improved peripheral oxygenation and a greater tendency toward stable high peripheral oxygen saturation levels. The residual benefit of supplemental oxygen therapy is further evidenced by higher oxygenation values during the final room-air interval.

It is noted that within normal clinical settings requiring supplementary oxygen, it would be unusual for flow rates to exceed 4 liters/minute. These results confirm efficient and predictable oxygen delivery of this hydrogel-adhesive nasal cannula medical device between flow rates of 2 and 4 liters/minute.

In some embodiments of the invention, the cannula example provides stable placement (i.e., no movement after placement), ease of application and removal and the tubing diameter is matched to oxygen wall mounting supplies. In some examples, the adherent medical device is designed as left or right pieces with the tubing emanating from the applicable side depending on clinical requirements. In these examples, the left and right designs may be color coded and are mirror images of each other.

In the description above, numerous specific details are set forth. However, it is understood that embodiments of the invention may be practiced without these specific details. For example, well-known equivalent components and elements may be substituted in place of those described herein, and similarly, well-known equivalent techniques may be substituted in place of the particular techniques disclosed. In other instances, well-known structures and techniques have not been shown in detail to avoid obscuring the understanding of this description.

Reference in the specification to “an embodiment,” “one embodiment,” “some embodiments,” or “other embodiments” means that a particular feature, structure, or characteristic described in connection with the embodiments is included in at least some embodiments, but not necessarily all embodiments. The various appearances of “an embodiment,” “one embodiment,” or “some embodiments” are not necessarily all referring to the same embodiments. If the specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

What is claimed is:

1. A skin adherent device comprising:
a first portion configured for coupling to a first portion of an anatomy;
a second portion configured for coupling to a second portion of the anatomy; and
a third portion configured for coupling to a third portion of the anatomy, wherein the second portion and the third portion are disposed on different sides of the skin adherent device, and the first portion, the second portion and the third portion comprise a tacky gel material.

2. The skin adherent device of claim 1, wherein the tacky gel material comprises one of hydrogel and hydrocolloid.

3. The skin adherent device of claim 2, wherein the first portion of the anatomy comprises an upper lip.

4. The skin adherent device of claim 3, wherein the second portion of the anatomy and the third portion of the anatomy comprise one of paranasal portions and cheek portions.

5. The skin adherent device of claim 4, further comprising:
a tubular bridge portion coupled to the first portion, wherein the tubular bridge portion including a pair of nasal prongs configured for delivering a gas to nasal passages of a user.

6. The skin adherent device of claim 5, wherein the tubular bridge portion only includes a single gas inlet.

7. The skin adherent device of claim 6, further comprising an anchor skin paddle configured for supporting a gas delivery tube coupled to the single gas inlet, wherein the anchor skin paddle comprises a skin adherent gel material.

8. The skin adherent device of claim 1, wherein the first portion, the second portion and the third portion are removably coupled to portions of a face having facial hair without causing discomfort to a user upon removing from the portions of the face having facial hair.

9. The skin adherent device of claim 7, wherein the gas delivery tube has an interior diameter less than an interior diameter of the tubular bridge portion, and the outer diameter of the delivery tube is equal to the diameter of the interior diameter of the tubular bridge for decelerating gas flow rate into the tubular bridge portion.

10. The skin adherent device of claim 6, wherein the tubular bridge portion includes an outlet tube configured for sampling gas.

11. The skin adherent device of claim 2, further comprising a nasal canopy portion configured for covering a user's nose.

12. The skin adherent device of claim 2, wherein the skin adherent device is configured for securing intravenous delivery lines.

13. The skin adherent device of claim 2, wherein the skin adherent device is impregnated with material for promoting one of antisepsis, anti-itch, anti-allergic, anti-inflammatory and anti-aging.

14. A cannula device comprising:
a first portion configured for coupling to an upper lip portion of an anatomy;
a second portion configured for coupling to a second portion of the anatomy; and
a third portion configured for coupling to a third portion of the anatomy, wherein the second portion and the third portion are disposed on different sides of the cannula device, and the first portion, the second portion and the third portion comprise a tacky gel material.

15. The cannula device of claim 14, wherein the second portion of the anatomy and the third portion of the anatomy comprise one of paranasal portions and cheek portions.

16. The cannula device of claim 15, further comprising:
a tubular bridge portion coupled to the first portion, wherein the tubular bridge portion including a pair of curved nasal prongs configured for delivering a gas to nasal passages of a user.

17. The cannula device of claim 16, wherein the tubular bridge portion only includes a single gas inlet.

18. The cannula device of claim 17, further comprising an anchor skin paddle configured for supporting a gas delivery tube coupled to the single gas inlet, wherein the anchor skin paddle comprises a skin adherent gel material.

19. The cannula device of claim 18, wherein the gas delivery tube and the tubular bridge portion have a same interior diameter for minimizing noise turbulence.

20. An apparatus comprising:
an anchoring device configured for coupling other devices to an anatomy, wherein the anchoring device comprises a tacky gel material.

21. The apparatus of claim 20, wherein the anchoring device comprises one of a cannula, an intravenous line anchor, a medical post-procedure patch, a continuous positive airway pressure (CPAP) device, and an electrode anchoring device.

22. The apparatus of claim 21, wherein the anchoring device is impregnated with a health promoting agent.
